(19) United States
(12) Patent Application Publication
(10) Pub. No.: US 2016/0095576 A1
(43) Pub. Date: Apr. 7, 2016

(54) DIAGNOSIS SUPPORTING APPARATUS AND DIAGNOSIS SUPPORTING METHOD

(71) Applicant: SAMSUNG ELECTRONICS CO., LTD., Suwon-si (KR)

(72) Inventor: Moon Ho PARK, Hwaseong-si (KR)

(73) Assignee: SAMSUNG ELECTRONICS CO., LTD., Suwon-si (KR)

(21) Appl. No.: 14/877,510

(22) Filed: Oct. 7, 2015

(30) Foreign Application Priority Data
Oct. 7, 2014 (KR) 10-2014-0135269

(51) Int. Cl. A61B 8/00; A61B 8/08; A61B 8/14; A61B 5/055; A61B 6/03

(52) U.S. Cl. CPC A61B 8/4245; A61B 5/055; A61B 6/032; A61B 8/14; A61B 8/5215

(57) ABSTRACT

Provided are a diagnosis supporting apparatus and a diagnosis supporting method that analyzes characteristics of medical examinations and supports diagnosis, in which examination characteristics of a practitioner may be analyzed by comparing the characteristics with a reference examination, thereby enabling accurate and efficient diagnosis. The diagnosis supporting apparatus includes: an examination path acquiring component configured to acquire an examination path of an examination device; an examination characteristic analyzer configured to analyze characteristics of the examination based on the acquired examination path by using reference examination information; and a diagnosis component configured to determine an analysis point based on the reference examination and the analyzed examination characteristics, and to obtain a diagnosis with respect to one or more images that relate to the analysis point on the examination path.

### CROSS-REFERENCE TO RELATED APPLICATION(S)

This application claims priority from Korean Patent Application No. 10-2014-0135269, filed on Oct. 7, 2014, in the Korean Intellectual Property Office, the disclosure of which is incorporated herein by reference in its entirety for all purposes.

### BACKGROUND

1. Field

Exemplary embodiments relate to a diagnosis supporting apparatus and diagnosis supporting method that analyzes characteristics of medical examinations and supports diagnosis.

2. Description of the Related Art

In sonography, ultrasound images may be acquired in real time so that structures and movements of body organs may be observed, and the bloodstream in blood vessels may be measured. Further, the sonography may be conducted rapidly without pain and without radioactive rays that are harmful to the human body, thereby facilitating easy and convenient diagnosis of diseases or determination of treatment progress.

While an ultrasound examination may be conducted in a simple manner, it is difficult to evaluate ultrasound images, which requires a good understanding of the human body and ultrasound imaging, as well as of various artifacts and errors that may occur during the ultrasound examination. Reliability of the ultrasound examination is highly dependent on the ability of medical practitioners that conduct the ultrasound examination.

However, since a general ultrasound examination method has not been standardized, examinations are conducted by a respective method preferred by each practitioner, and examination results are affected by each practitioner's individual environment. In addition, as examination accuracy depends on the respective competence of each practitioner, the examinations are required to be checked objectively and systematically in order to prevent mistakes of expert practitioners and trainee doctors, and to improve examination efficiency of practitioners.

### SUMMARY

In one general aspect, there is provided a diagnosis supporting apparatus, including: an examination path acquiring component configured to acquire an examination path of an examination device; an examination characteristic analyzer configured to analyze at least one characteristic of an examination based on the acquired examination path by using reference examination information; and a diagnosis component configured to determine an analysis point based on the reference examination information and the analyzed at least one examination characteristic, and to obtain a diagnosis based on at least one image that relates to the determined analysis point.

The at least one examination characteristic may include at least one from among an examination path, a moving distance of the examination device, a moving speed in at least one section, an average moving speed, a period of staying in at least one area, an examination frequency in at least one area, a path difference between the acquired examination path and the reference examination information, and a difference between an examination frequency of the examination path with respect to at least a first section and an examination frequency that relates to the reference examination information with respect to the at least first section.

The examination characteristic analyzer may be further configured to convert the examination path into a graphic image of a heat distribution type by using at least one of the at least one examination characteristic.

The diagnosis component may be further configured to compare the acquired examination path with a reference path that relates to the reference examination information, and to determine the analysis point as a section in which a difference between the acquired examination path and the reference path exceeds a predetermined threshold.

The diagnosis component may be further configured to calculate a distance between at least one point on the examination path and a predetermined point within the analysis point, and based on the calculated distance, to determine a point from among the at least one point on the examination path in order to obtain a diagnosis based on an image that relates to the determined point.

The diagnosis component may be further configured to determine, as the analysis point, at least one from among a section in which a moving speed exceeds a predetermined first threshold, a section in which an average speed is greater than a predetermined second threshold, a section in which a period of staying in at least one area is shorter than a predetermined third threshold, and a section in which an examination frequency is lower than a predetermined fourth threshold.

The diagnosis component may be further configured to extract a point at which respective examination paths intersect in the analysis point, and to obtain a diagnosis based on an image that relates to the extracted point.

The diagnosis supporting apparatus may further include an output device configured to output at least one from among the at least one examination characteristic, the analysis point, and a result of the obtained diagnosis.

The output device may be further configured to visually output, on a screen, the at least one examination characteristic based on a difference between the acquired examination path and the reference examination information.

The diagnosis supporting apparatus may further include a reference examination selector configured to automatically select a first reference examination from among a plurality of reference examinations based on at least one from among an input received from a practitioner and a plurality of characteristics of the practitioner that include a competence of the practitioner.

In another general aspect, there is provided a diagnosis supporting method, including: acquiring an examination path of an examination device; analyzing at least one characteristic of an examination based on the acquired examination path by using reference examination information; determining an analysis point based on the reference examination information and the analyzed at least one examination characteristic; and obtaining a diagnosis based on at least one image that relates to the determined analysis point.

The at least one examination characteristic may include at least one from among an examination path, a moving distance of the examination device, a moving speed in at least one section, an average moving speed, a period of staying in at least one area, an examination frequency in at least one area, a path difference between the acquired examination path and the reference examination information, and a difference between an examination frequency of the examination path with respect to at least a first section and an examination frequency that relates to the reference examination information with respect to the at least first section.

The analyzing the at least one examination characteristic may include converting the examination path into a graphic image of a heat distribution type by using at least one from among the at least one examination characteristic.

The determining the analysis point may include comparing the acquired examination path with a reference path that relates to the reference examination information, and determining the analysis point as a section in which a difference between the acquired examination path and the reference path exceeds a predetermined threshold.

The obtaining the diagnosis may include: calculating a distance between at least one point on the examination path and a predetermined point within the analysis point; based on the calculated distance, determining a point from among the at least one point on the examination path; and obtaining a diagnosis based on an image that relates to the determined point.

The determining the analysis point may include determining, as the analysis point, at least one from among a section in which a moving speed exceeds a predetermined first threshold, a section in which an average speed is greater than a predetermined second threshold, a section in which a period of staying in at least one area is shorter than a predetermined third threshold, and a section in which an examination frequency is lower than a predetermined fourth threshold.

The obtaining the diagnosis may include extracting a point at which respective examination paths intersect in the analysis point, and obtaining a diagnosis based on an image that relates to the extracted point.

The diagnosis supporting method may further include outputting at least one from among the at least one examination characteristic, the analysis point, and a result of the obtaining the diagnosis.

The outputting may include visually outputting, on a screen, the at least one examination characteristic based on a difference between the acquired examination path and the reference examination information.

The diagnosis supporting method may further include automatically selecting a first reference examination from among a plurality of reference examinations based on at least one from among an input received from a practitioner and a plurality of characteristics of the practitioner that include a competence of the practitioner.

### BRIEF DESCRIPTION OF THE DRAWINGS

FIG. 1 is a block diagram illustrating an example of a diagnosis supporting apparatus, according to an exemplary embodiment.

FIG. 2 is a diagram illustrating an example of images at an analysis point on an examination path.

FIG. 3 is a diagram illustrating an example of determining an analysis point by using graphic images of a heat distribution type.

FIG. 4 is a diagram illustrating an example of supporting diagnosis of an analysis point by using images at an intersection point.

FIG. 5 is a diagram illustrating an example of a table showing the types of reference examination in the ultrasound examination.

FIG. 6 is a flowchart illustrating an example of a diagnosis supporting method in consideration of examination characteristics of practitioners, according to an exemplary embodiment.

FIG. 7 is a flowchart illustrating an example of a diagnosis supporting method in consideration of examination characteristics of practitioners, according to another exemplary embodiment.

Throughout the drawings and the detailed description, unless otherwise described, the same drawing reference numerals will be understood to refer to the same elements, features, and structures. The relative size and depiction of these elements may be exaggerated for clarity, illustration, and convenience.

### DETAILED DESCRIPTION

The present inventive concept will now be described more fully with reference to the accompanying drawings, in which exemplary embodiments are shown. The present inventive concept may, however, be embodied in many different forms, and should not be construed as being limited to the exemplary embodiments set forth herein; rather, these exemplary embodiments are provided so that the present disclosure will be thorough and complete, and will fully convey the present inventive concept to those skilled in the art. Therefore, the scope of the present disclosure is defined not by the detailed description but by the appended claims. Throughout the drawings and the detailed description, unless otherwise described, the same drawing reference numerals will be understood to refer to the same elements, features, and structures.

Hereinafter, the diagnosis supporting apparatus and the diagnosis supporting method will be described in detail with reference to the accompanying drawings.

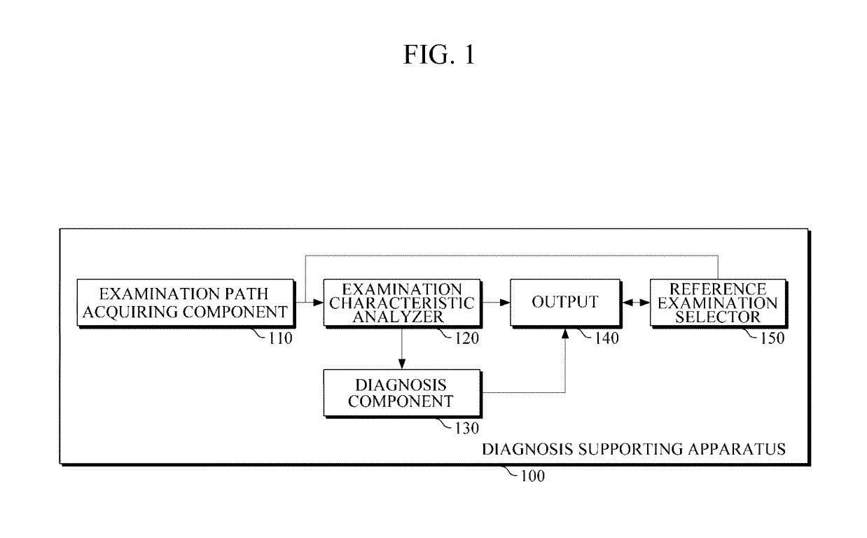

FIG. 1 is a block diagram illustrating an example of a diagnosis supporting apparatus, according to an exemplary embodiment.

The diagnosis supporting apparatus 100 includes an examination path acquiring component (also referred to herein as an “examination path acquirer”) 110, an examination characteristic analyzer 120, a diagnosis component (also referred to herein as a “diagnoser”) 130, an output (also referred to herein as an “output device”) 140, and a reference examination selector 150.

The examination path acquiring component 110 acquires, from a medical image diagnosis device, an examination path of an examination device that is configured to perform an examination of a subject. The medical image diagnosis device may be an ultrasound imaging device in which ultrasound images are acquired in real time by pressing a probe to a patient's body portion to be examined. For convenience of explanation, the following description will be made by using a real-time ultrasound imaging device. However, the exemplary embodiments are not limited thereto, and any of various medical imaging devices, such as a magnetic resonance imaging (MRI) device or a computerized tomography (CT) device, may also be used.

A practitioner generally performs examinations by pressing a probe to a portion to be examined of a subject and by moving the probe in a winding manner. In some cases, a probe stays at a specific part for an extended period of time if necessary so as to examine the specific part more thoroughly. As described above, in the examination using ultrasound images, examination characteristics may vary based on the competence, gender, age, and the like, of practitioners, which may result in various and different examination paths.

The examination characteristic analyzer 120 analyzes one or more examination characteristics of the acquired examination path by using reference examination information.

The reference examination information may include information that relates to any of various reference examinations, including examinations generated according to a standard body model, a subject's individual characteristics, characteristics of each body portion to be examined (e.g., abdomen, breast, liver, etc.), the competence of practitioners, average examination records of medical institutions, examination categories, and the like. Further, the reference examination information may include information on the characteristics of a subject to be examined, the competence of practitioners, a reference path according to average examination records of medical institutions, a moving distance in the reference examination, a moving speed in each section of the body being examined, an average moving speed, a period of staying in each area being examined, an examination frequency in each area, a salient region, and the like. However, the examples are merely illustrative, and the reference examination information is not limited thereto.

The examination characteristic analyzer 120 may analyze one or more characteristics of an examination conducted by a practitioner by comparing the reference examination information to information on the acquired examination path. The characteristics of an examination may include an examination path, a moving distance of an examination device, a moving speed in each section, an average moving speed, a period of staying in each area, an examination frequency in each area, a difference in paths between the examination path and the reference examination information, and a difference in examination frequencies in each section between the examination path and the reference examination information.

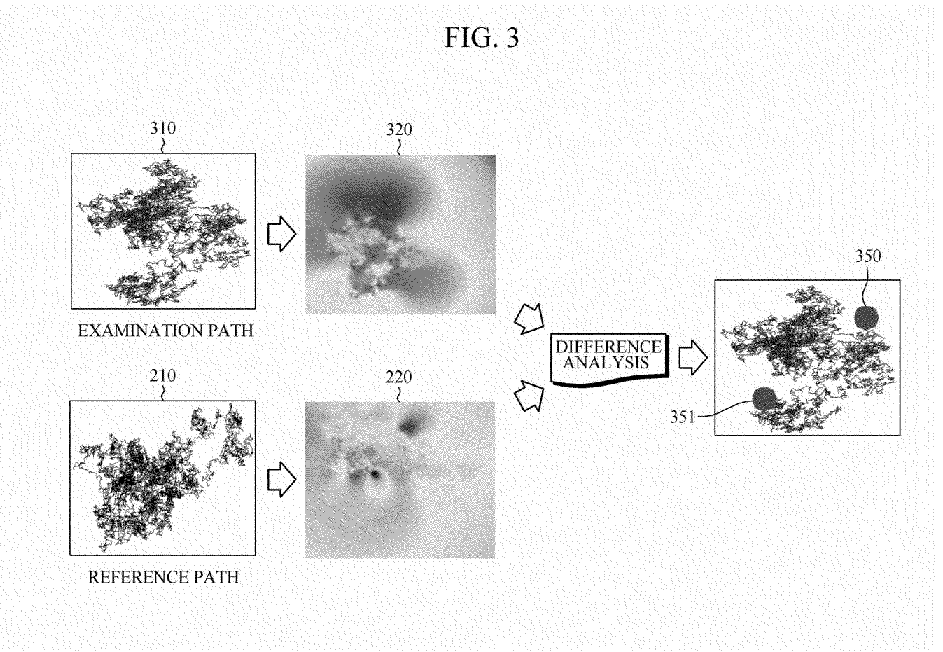

In one exemplary embodiment, the examination characteristic analyzer 120 may identify the types of examination conducted by a practitioner, and may analyze examination characteristics to determine a quality of an examination. For example, by using one or more of the examination characteristics, the examination characteristic analyzer 120 may convert an examination path into a graphic image of a specific type, such as a heat distribution type, or a contour type, which will be described below with reference to FIG. 3.

In the case in which the reference examination is an average examination of medical institutions, the examination characteristic analyzer 120 may analyze, for each category, characteristics of examinations conducted by a practitioner by comparing a moving speed in each section of an examination performed by a practitioner to a moving speed in each section of an average examination of medical institutions. Further, the examination characteristic analyzer 120 may determine a quality of an examination conducted by a practitioner by combining analysis results of other characteristics. The analysis may be applied in the same manner to other characteristics or other exemplary embodiments selected as a reference examination.

Based on the reference examination and the analyzed examination characteristics, the diagnosis component 130 may determine an analysis point, and obtain a diagnosis based on images that relate to the analysis point on an examination path. To this end, the diagnosis component 130 may compare an examination path to a reference path of the reference examination in order to determine a section as being a diagnosis point at which in the section, the difference between the examination path and the reference path is greater than a predetermined threshold. For example, the diagnosis component 130 may determine a diagnosis point by using a graphic image of a heat distribution type analyzed based on examination characteristics, which will be described below with reference to FIG. 3.

The diagnosis component 130 may analyze images that relate to a diagnosis point, in which the analysis of images may include determining a region of interest, calculating a risk level as a lesion probability, determining of an amount of muscle or an amount of body fat, and the like.

The diagnosis component 130 may determine a diagnosis point based on the difference between a reference path of the reference examination and an examination path, in which the comparison of paths may be performed on the basis of dots, lines, and sides according to requirements of examinations.

In one exemplary embodiment, there may be salient points, for which images are necessary to acquire, depending on the characteristics of each body organ to be examined. By conducting a point-to-point comparison, the diagnosis component 130 may obtain diagnoses based on images of salient points on an examination path that correspond to several predetermined salient points on a reference path. In this case, the diagnosis component 130 may determine salient points on a reference path and sections on an examination path that correspond to the salient points to be analysis points. In the case where there are points on an examination path that correspond to salient points on a reference path within analysis points, the diagnosis component 130 obtains diagnoses based on images at those points, and in the case where there are no points that correspond to salient points on a reference path, the diagnosis component 130 determines an adjacent point in order to obtain a diagnosis based on an image at that point. In one exemplary embodiment, the determination of an adjacent point may include determining a point on an examination path that is closest to a predetermined salient point on a reference path.

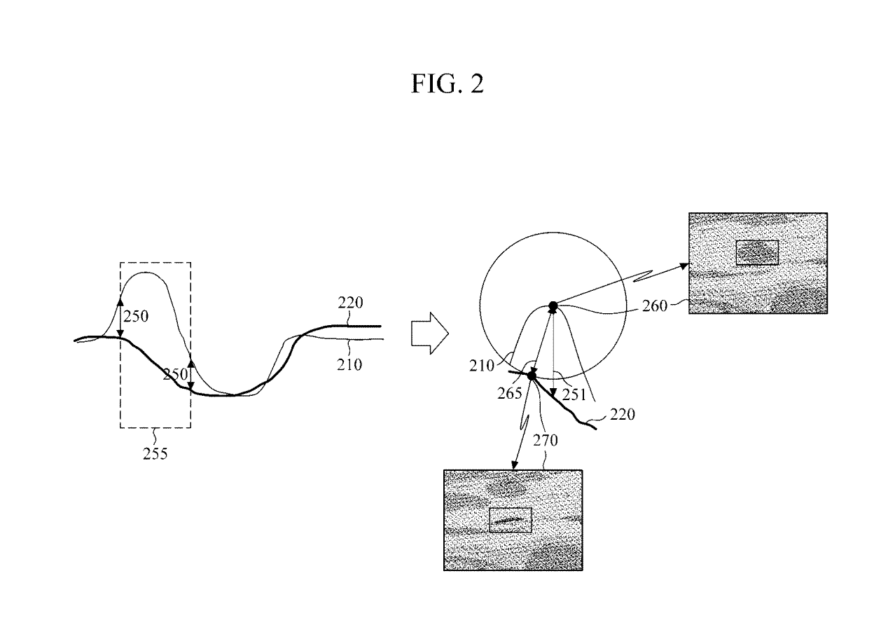

In another exemplary embodiment, a path of a reference examination and a path of an examination conducted by a practitioner may be similar to each other. In this case, the diagnosis component 130 may compare the two paths in order to determine a section having a difference that is greater than a predetermined threshold to be an analysis point. Further, the diagnosis component 130 calculates a distance between one or more points on an examination path and a specific point within an analysis point, and based on the calculated distance, determines a point on an examination path that is closest to a point on a reference path in order to obtain a diagnosis based on an image of the determined point, which will be described below with reference to FIG. 2.

In still another exemplary embodiment, the diagnosis component 130 may determine, as an analysis point, a section in which a moving speed is greater than a predetermined threshold, a section in which an average speed is greater than a predetermined threshold, a section in which a period of staying in at least one area is shorter than a predetermined threshold, and a section in which an examination frequency is lower than a predetermined threshold, among examination characteristics. In this case, the thresholds may be values that are predetermined by using reference examination information.

For example, in the case where a moving speed in each section of an imaging device is relatively high or an average speed is relatively high, the examination path acquiring component 110 may miss a section and may not acquire necessary images. Further, in the case where a period of staying in an area that corresponds to a salient point which includes a region of interest is relatively short or an examination frequency is relatively low, sufficient data may not obtained to analyze and diagnose images. In this case, the diagnosis component 130 may determine the above section to be an analysis point so as to supplement a diagnosis.

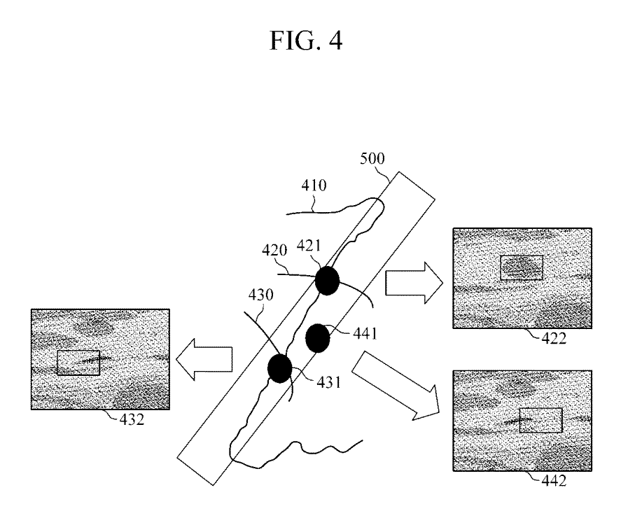

For example, the diagnosis component 130 may determine, as an analysis point, a section in which a moving speed of an imaging device is greater than a predetermined threshold, and may extract a point at which respective examination paths intersect within an analysis point in order to obtain a diagnosis based on images of the extracted point, which will be described in detail with reference to FIG. 4.

Further, the diagnosis supporting apparatus 100 includes an output 140 that outputs characteristics of an analyzed examination, analysis points, and diagnosis results. Further, the output 140 may visually output examination characteristics based on the difference between the examination path and the reference examination information.

Various outputting methods may be used by the output 140 to output examination characteristics analyzed in terms of the types and quality of an examination conducted by a practitioner. In one exemplary embodiment, the examination characteristic analyzer 120 may convert an examination path into graphic image data of a heat distribution type by using one or more of the examination characteristics, and the output 140 may output the data. In this case, the graphic image of a heat distribution type may visually represent an examination frequency of each area. For example, the examination frequency may be displayed by using a color index, in which the output 140 may display an area in red by considering that the area has a relatively high examination frequency with a long period of staying in that area, and with examination paths being concentrated therein due to a thorough examination. Further, the output 140 may display an area in yellow by considering that the area has a relatively low examination frequency with a short period of staying in that area and with examination paths being dispersed. The output 140 may display other areas in a value between red and yellow by determining a threshold of each section according to respective examinations. The colors of red and yellow are selected at random for convenience of explanation, and other colors may also be used to output a graphic image of a heat distribution type.

In another exemplary embodiment, the examination characteristic analyzer 120 may convert an examination path into a graphic image of a contour type by using one or more of examination characteristics, and the output 140 may output the data. When the examination path is acquired by using an ultrasound imaging device that varies based on the characteristics of an organ to be examined, the examination path may include data regarding depth. The output 140 may output graphic image data of a contour type by including data regarding depth in characteristics of an examination conducted by a practitioner. For example, a threshold may be determined according to depths, and each section may be displayed by using a different color index that varies based on depths, in which among examination paths, a relatively deep examination path may be displayed in red, and a relatively shallow examination path may be displayed in yellow. The colors of red and yellow are selected at random for convenience of explanation, and other colors may also be used to output a graphic image of a heat distribution type.

In still another exemplary embodiment, the output 140 may output an analysis point. For example, the diagnosis component 130 may compare a reference path included in reference examination information with an examination path included in characteristics of an examination conducted by a practitioner, in order to determine a section having a difference that is greater than a threshold between the examination path and the reference examination path to be an analysis point, and the output 140 may output the determined analysis point on a screen, and may output results of diagnosis of the analysis point performed by the diagnosis component 130.

Further, based on the difference between the examination path and the reference examination information, the output 340 may visually output characteristics of an examination. By comparing a reference path included in reference examination information with an examination path included in characteristics of an examination conducted by a practitioner, a section may be determined based on a difference obtained upon the comparison, and the output 140 may visually output the examination path in a heat distribution type or a contour type. For example, even when the difference is not significantly above a threshold, the output 140 may visually output the difference between a reference examination and an examination path. Further, the output 140 may visually output a moving distance of an examination device, a moving speed in each section, an average moving speed, a period of staying in each area, an examination frequency in each area, and the like, based on the difference of each category. In this case, the output 140 may output only the difference, or may output the difference by overlaying the difference on an examination path.

In addition, the diagnosis supporting apparatus 100 may further include a reference examination selector 150, which may automatically select a reference examination from among a plurality of reference examinations based on a practitioner's characteristics that include competence, and/or based on a practitioner's input.

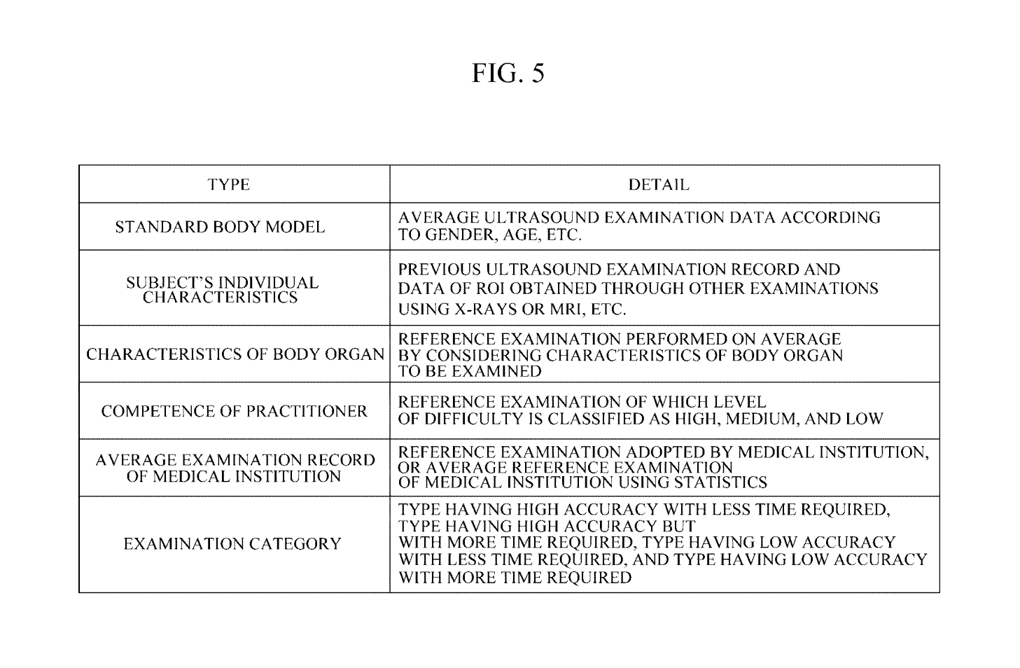

The reference examination selector 150 may select a reference examination before a practitioner conducts an examination, and may select/re-select a reference examination after a practitioner conducts an examination. The reference examination may be selected based on the purpose and need of examinations, and the types of the reference examination will be described below with reference to FIG. 5.

FIG. 2 is a diagram illustrating an example of images at an analysis point on an examination path.

A left portion of FIG. 2 illustrates an examination path 220 and a reference path 210 on the same coordinate axes. A diagnosis component of the diagnosis supporting apparatus 100 may compare the examination path 220 with the reference path 210 of a reference examination in order to determine a section in which there is a difference 250 therebetween that exceeds a predetermined threshold as an analysis point 255. The diagnosis component may perform a more specific diagnosis with respect to the determined analysis point 255.

A right portion of FIG. 2 illustrates an example of extracting an image on an examination path and obtaining a diagnosis based on the extracted image. The diagnosis component may extract a specific image within the diagnosis point 255 in any of various manners, among which is included a method of determining a section with a significant difference between the examination path 220 and the reference path 210, and extracting some points 270 on the examination path 220 that are closest to each other in order to obtain a diagnosis based on an image of the extracted point 270 will be described with reference to the right portion of FIG. 2.

Referring to the right figure of FIG. 2, the diagnosis component calculates a distance between the examination path 220 and the reference path 210 and determines a point in a section 251 having the biggest difference therebetween. Further, the diagnosis component calculates the point 270 on the examination path that is closest to a point 260 on the reference path.

For example, the diagnosis component may calculate a distance between the point 260 on the reference path and a plurality of points on the examination path 220. Further, the diagnosis component may extract the point 270 on the examination path that is determined to be closest to the point 260 on the reference path based on the calculation, and may obtain a diagnosis based on an image of the extracted point 270.

In another example, by using a circle for which the point 260 on the reference path is at the center thereof, the diagnosis component may calculate whether the point 270 on the examination path is included in the circle. Referring to the right figure of FIG. 2, the diagnosis component may form a circle having a radius 265 with the point 260 on the reference path being at the center thereof, and the point 270 on the examination path may be included in the circle. In the case where no point on the examination path is included in the circle for which the point 260 on the reference path is at the center thereof, the diagnosis component gradually increases the size of the radius 265 in order to obtain a point of intersection on the reference path. Further, in the case where a plurality of points 270 on the examination path are included in the circle for which the point 260 on the reference path is at the center thereof, the diagnosis component selects a point on the reference path that is closest to the examination path, or may extract images of all the plurality of points.

Various exemplary embodiments, at the diagnosis component, of determining a specific point within the analysis point, and extracting an image of a point on the examination path that is closest to the specific point may be implemented, such that the present inventive concept is not limited thereto.

FIG. 3 is a diagram illustrating an example of determining an analysis point by using graphic images of a heat distribution type.

The diagnosis supporting apparatus 100 may convert an examination path into a graphic image of a heat distribution type by using one or more of examination characteristics. Referring to FIG. 3, the left upper drawing of FIG. 3 illustrates an examination path 310. In the case in which the examination path 310 is complicated, the diagnosis component may use a graphic image of a heat distribution type, rather than analyzing a movement of the examination path 310 itself, in order to determine an analysis point.

An examination characteristic analyzer of the diagnosis supporting apparatus 100 may quantify data, such as a period of staying in each area or an examination frequency of each area, from among characteristics of an examination conducted by a practitioner, and may convert the data into a graphic image of a heat distribution type. The output may display the data using a color index and may output a heatmap 320 that relates to an examination path, and similarly, the output may also output a heatmap 220 that relates to the reference path 210.

Referring to FIG. 3, when an examination characteristic analyzer of the diagnosis supporting apparatus 100 analyzes an examination path using the heatmap 320, the examination characteristic analyzer may output an area in red, or in a specific color that has relatively high brightness or that is relatively dark on a grayscale, by considering that some middle upper portion of the area and some middle lower portions of the area have a high examination frequency with a long period of staying, and with examination paths being concentrated therein due to a thorough examination. By contrast, the output 140 may output an area in yellow, or in a color that has relatively low brightness or is relatively light on a grayscale, by considering that a right portion of the area has a relatively low examination frequency. Similarly, when the heatmap 220 regarding the reference path 210 is output, the examination characteristic analyzer of the diagnosis supporting apparatus 100 may output an area in orange by considering that in the area, the reference path 210 is spread over a relatively wide area with respect to a central portion and has a relatively high examination frequency.

Upon analyzing the difference between the heatmap 320 of the examination path and the heatmap 220 of the reference path, the diagnosis component of the diagnosis supporting apparatus 100 may determine two analysis points 350 and 351 as illustrated in the right drawing of FIG. 3. Referring to the right drawing of FIG. 3, the output may output the analysis points 350 and 351 having a difference that is above a threshold by overlaying the analysis points 350 and 351 on the examination path 310.

FIG. 4 is a diagram illustrating an example of supporting diagnosis of an analysis point by using images at an intersection point. Referring to FIG. 4, an examination path 410 corresponds to a reference path 500 of a reference examination, and there are two examination paths 420 and 430. In the ultrasound examination, images may be acquired easily such that a practitioner may perform an examination of a salient body portion more thoroughly, which leads to many paths intersecting during the examination.

Referring to FIG. 4, for example, the reference path 500 of a reference examination may be predetermined to be a salient point, at which it is necessary that images be acquired for examination. However, in the case where a medical image diagnosis device moves too rapidly while a practitioner performs an examination, images required for the diagnosis may not be acquired in a section on the examination path 410 that corresponds to the reference path. However, even in this case, the diagnosis component may support obtaining a diagnosis based on the image, in which the diagnosis component may determine, as the analysis point 500, a section in which a moving speed of a medical image diagnosis device that is used by a practitioner to examine a subject is greater than a threshold. The threshold may be calculated based on a moving speed in each section of the reference examination.

Further, the diagnosis component may determine whether there are other intersection points in the analysis point 500. Referring to FIG. 4, there are other examination path 420 and 430 that intersect in the analysis point 500, and the diagnosis component extracts the intersection points 421, 431, and 441 in order to obtain images 422, 432, and 443 of the extracted points. In this case, by referring to FIG. 4, the intersection points 421 and 431 are points on the examination path 410 and the intersection point 441 is an adjacent point in the analysis point. Images of the intersection points 421 and 431 that are acquired by the diagnosis component may be diagnosed as images on the examination path 410 that corresponds to the reference path in the analysis point 500 or as an adjacent image 442.

FIG. 5 is a diagram illustrating an example of a table showing the types of reference examination in the ultrasound examination. The diagnosis supporting apparatus 100 compares a reference examination with a path of an examination conducted by a practitioner. Various exemplary embodiments of the reference examination may be implemented, and reference examinations may be selected based on the respective purposes of examinations.

Referring to FIG. 5, the reference examinations may be separated according to a standard body model, a subject's individual characteristics, characteristics of each body portion to be examined, average examination records of medical institutions, and examination categories.

As one type of the reference examination, the standard body model may be based on an average ultrasound examination data according to a gender, an age, and the like, of a subject. As another type of the reference examination, a subject's individual characteristics may be used. By using a previous ultrasound examination record of a subject as a reference examination, and comparing the reference examination with a current examination, a subject's body change according to elapsed time may be identified. In other cases for which there are data such as locations or coordinates of regions of interest obtained from other examinations using X-rays or MRI, a region of interest of a subject may be displayed in the standard body model and may be selected as a reference examination.

Another type of the reference examination relates to characteristics of each body portion to be examined. In the ultrasound examination, various body organs may be examined, including upper body parts, such as liver, cholecyst (gall bladder), pancreas, spleen, kidney, and the like; pelvic cavity organs, such as bladder, womb, ovary, prostate, and the like; and thyroid, breast, scrotum, musculoskeletal organs, heart, and the like. In this case, each organ has its own respective characteristics that are required for the ultrasound examination, such that a reference examination that is conducted on average may be selected based on the respective characteristics of each organ.

Another type of the reference examination relates to a practitioner's competence. For example, by determining a wide range of a threshold that is used to evaluate quality of a reference examination to be compared, or by determining an easier reference path of a reference examination, a level of difficulty of a reference examination may be determined. The reference examination is data to be compared with characteristics of an examination conducted by a practitioner, and the level of difficulty may be adjusted if necessary.

Another type of the reference examination relates to average examination records of medical institutions. A reference examination adopted by a medical institution or an average reference examination of a medical institution using statistics may be used as a reference examination.

Further, another type of the reference examination relates to examination categories. For example, reference examinations may be separated into a type having a relatively high accuracy with less time required; a type having a relatively high accuracy but with more time required; a type having a relatively low accuracy with less time required; and a type having a relatively low accuracy with more time required.

The above examples are merely exemplary and other various types of reference examination may also be used, depending on the purpose and need of examinations.

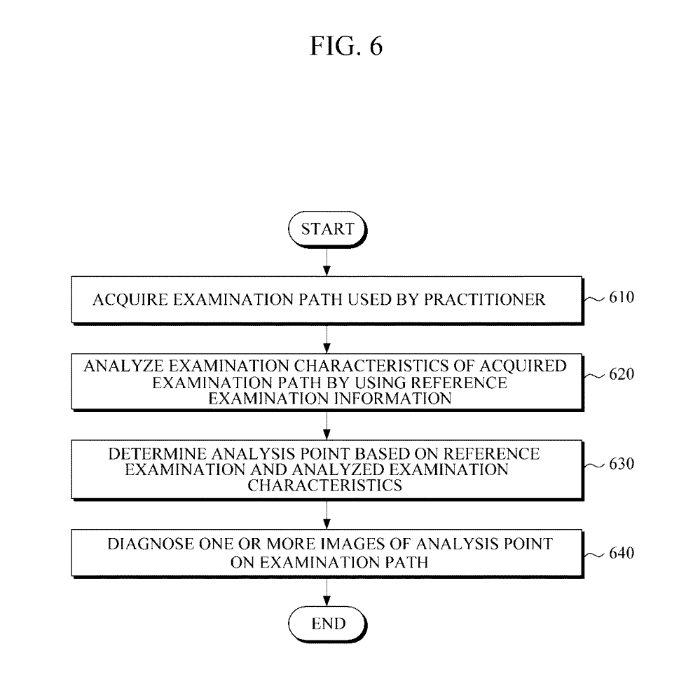

FIG. 6 is a flowchart illustrating an example of a diagnosis supporting method in consideration of examination characteristics of practitioners, according to an exemplary embodiment. The diagnosis supporting method may be performed in consideration of examination characteristics of a practitioner by using the diagnosis supporting apparatus 100 illustrated in FIG. 1.

The diagnosis supporting apparatus 100 acquires, from a medical image diagnosis device, an examination path used by a practitioner to examine a subject to be examined in operation 610. In general, a practitioner performs an examination by pressing a probe against the subject and by moving the probe. In some cases, a probe stays at a specific part of the subject for an extended period of time if necessary so as to examine the specific part more thoroughly. As described above, in the examination using ultrasound images, examination characteristics may vary based on one or more of the competence, gender, age, and the like, of practitioners, which may result in various and different examination paths.

Subsequently, in operation 620, the diagnosis supporting apparatus 100 may analyze examination characteristics of the acquired examination path by using reference examination information.

The diagnosis supporting apparatus 100 may compare the reference examination information with the acquired examination path to analyze characteristics of an examination conducted by a practitioner. In this case, the characteristics of an examination may include one or more of an examination path, a moving distance of an examination device, a moving speed in each section, an average moving speed, a period of staying in each area, an examination frequency in each area, a difference in paths between the examination path and the reference examination information, and a difference in examination frequencies in each section between the examination path and the reference examination information.

In one exemplary embodiment, the diagnosis supporting apparatus 100 may identify the types of examination conducted by a practitioner, and may analyze examination characteristics to determine a quality of an examination. For example, by using one or more of the examination characteristics, the diagnosis supporting apparatus 100 may convert an examination path into a graphic image of a specific type, such as a heat distribution type, or a contour type, which is described above with reference to FIG. 3.

In the case where the reference examination is an average examination of medical institutions, the diagnosis supporting apparatus 100 may analyze, for each category, characteristics of examinations conducted by a practitioner by comparing a moving speed in each section of an examination performed by a practitioner to a moving speed in each section of an average examination of medical institutions. Further, the diagnosis supporting apparatus 100 may determine a quality of an examination conducted by a practitioner by combining analysis results of other characteristics. The analysis may be applied in the same manner to other characteristics or other exemplary embodiments selected as a reference examination.

Then, in operation 630, the diagnosis supporting apparatus 100 determines an analysis point based on the reference examination and the analyzed examination characteristics. To this end, the diagnosis supporting apparatus 100 may compare an examination path to a reference path of the reference examination in order to determine a section to be a diagnosis point, such that in the determined section, the difference between the examination path and the reference path exceeds a predetermined threshold. For example, the diagnosis component 130 may determine a diagnosis point by using a graphic image of a heat distribution type analyzed based on examination characteristics.

The diagnosis supporting apparatus 100 may determine a diagnosis point based on the difference between a reference path of the reference examination and an examination path, in which the comparison of paths may be performed on the basis of dots, lines, and sides according to requirements of examinations.

In one exemplary embodiment, there may be salient points, for which there is a necessity that corresponding images be acquired, based on the characteristics of each body organ to be examined. By conducting a point-to-point comparison, the diagnosis supporting apparatus 100 may obtain one or more diagnoses based on images of salient points on an examination path that correspond to several predetermined salient points on a reference path. In this case, the diagnosis supporting apparatus 100 may determine salient points on a reference path and sections on an examination path that correspond to the salient points to be analysis points.

In another exemplary embodiment, a path of a reference examination and a path of an examination conducted by a practitioner by using the diagnosis supporting apparatus 100 may be similar to each other. In this case, the diagnosis supporting apparatus 100 may compare the two paths in order to determine a section having a difference that is greater than a threshold to be an analysis point.

In still another exemplary embodiment, the diagnosis supporting apparatus 100 may determine, as an analysis point, a section in which a moving speed is greater than a threshold, a section in which an average speed is greater than a threshold, a section in which a period of staying in each area is shorter than a threshold, and a section in which an examination frequency is lower than a threshold, among examination characteristics. In this case, the thresholds may be values that are predetermined by using reference examination information.

For example, in the case in which a moving speed in each section of an imaging device is relatively high or an average speed is relatively high at the diagnosis supporting apparatus 100, the diagnosis supporting apparatus 100 may miss a section and may thus fail to acquire necessary images. Further, in the case in which a period of staying in an area that is a salient point which includes a region of interest is relatively short or an examination frequency is relatively low, an amount of data that is obtained may be insufficient for a purpose of analyzing images and obtaining a diagnosis based on the images by the diagnosis supporting apparatus 100. In this case, the diagnosis supporting apparatus 100 may determine the above section to be an analysis point so as to supplement a diagnosis.

Then, in operation 640, the diagnosis supporting apparatus 100 obtains a diagnosis based on an image of an analysis point on an examination path.

The diagnosis supporting apparatus 100 may analyze images of a diagnosis point, in which the analysis of images may include determining a region of interest, calculating a risk level as a lesion probability, determining of the muscle or the body fat, and the like.

In one exemplary embodiment, in the case in which there are points on an examination path that correspond to salient points on a reference path within analysis points, the diagnosis supporting apparatus 100 obtains one or more diagnoses based on corresponding images at those points, and in the case in which there are no points that correspond to salient points on a reference path, the diagnosis supporting apparatus 100 determines an adjacent point in order to obtain a diagnosis based on an image at that point. For example, the determination of an adjacent point at the diagnosis supporting apparatus 100 may include determining a point on an examination path that is closest to a predetermined salient point on a reference path.

In another exemplary embodiment, the diagnosis supporting apparatus 100 calculates a distance between one or more points on an examination path and a specific point within an analysis point, and based on the calculated distance, determines a point on an examination path that is closest to a point on a reference path in order to obtain a diagnosis based on an image of the determined point.

In still another exemplary embodiment, the diagnosis supporting apparatus 100 may extract a point where examination paths intersect within an analysis point, and may obtain a diagnosis based on an image of the extracted point.

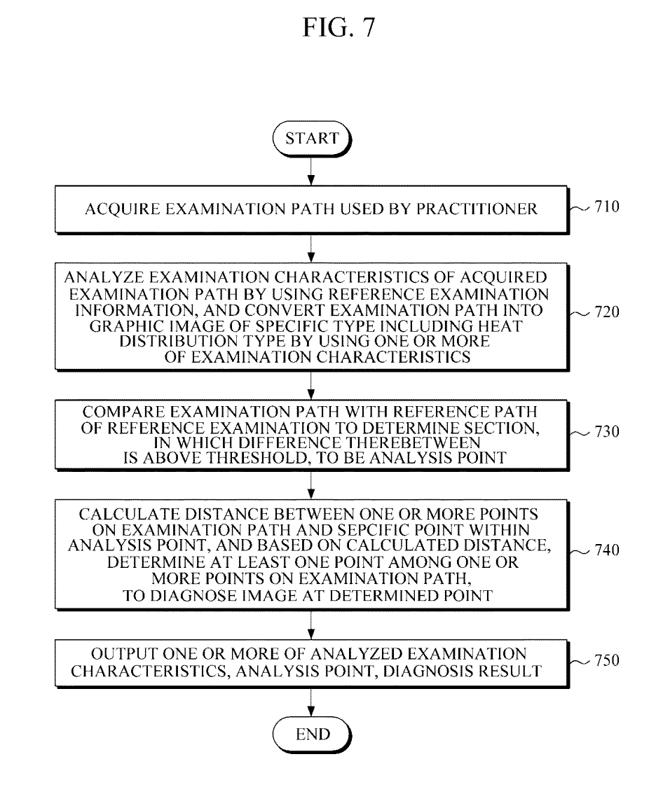

FIG. 7 is a flowchart illustrating an example of a diagnosis supporting method in consideration of examination characteristics of practitioners, according to another exemplary embodiment.

First, in operation 710, the diagnosis supporting apparatus 100 acquires, from a medical image diagnosis device, an examination path used by a practitioner to examine a subject to be examined.

Subsequently, in operation 720, the diagnosis supporting apparatus 100 may analyze examination characteristics on an examination path that are acquired by using the reference examination information, and by using one or more of the examination characteristics, the diagnosis supporting apparatus 100 may convert an examination path into a graphic image of a specific type, including a heat distribution type.

The diagnosis supporting apparatus 100 may quantify data, such as a period of staying in each area or an examination frequency of each area, of characteristics of an examination conducted by a practitioner, and may convert the data into a graphic image (heatmap) of a heat distribution type in operation 720.

Then, in operation 730, the diagnosis supporting apparatus 100 may compare an examination path with a reference path of a reference examination, and may determine a section in which a difference therebetween exceeds a threshold to be an analysis point.

For example, upon analyzing the difference between the heatmap of the examination path and the heatmap of the reference path, the diagnosis supporting apparatus 100 may determine a section in which a difference therebetween exceeds a threshold to be an analysis point.

Next, in operation 740, the diagnosis supporting apparatus 100 calculates a distance between one or more points on an examination path and a specific point within an analysis point, and based on the calculated distance, determines a point on an examination path that is closest to a point on a reference path in order to obtain a diagnosis based on an image of the determined point.

For example, the diagnosis supporting apparatus 100 may calculate a distance between the center of the analysis points 350 and 351 and a plurality of points on the examination path, may determine a point on the examination path that is determined to be closest to a point on the reference path based on the calculation, and may obtain a diagnosis based on an image of the determined point.

Then, in operation 750, the diagnosis supporting apparatus 100 may output analyzed examination characteristics, analysis points, and diagnosis results. Further, the diagnosis supporting apparatus 100 may visually output examination characteristics based on the difference between the examination path and the reference examination information. Various outputting methods may be used by diagnosis supporting apparatus 100 to output examination characteristics analyzed in terms of the types and quality of an examination conducted by a practitioner.

In one exemplary embodiment, the diagnosis supporting apparatus 100 may output graphic image data of a heat distribution type that is converted from an examination path by using one or more of the analyzed examination characteristics. In this case, the graphic image of a heat distribution type may visually represent an examination frequency of each area. For example, the examination frequency may be displayed by using a color index, in which the outputting in operation 750 may include displaying an area in red by considering that the area has a relatively high examination frequency with a relatively long period of staying, and with examination paths being concentrated therein due to a thorough examination. Further, the outputting in operation 750 may include displaying an area in yellow by considering that the area has a relatively low examination frequency with a relatively short period of staying and with examination paths being dispersed. The outputting in operation 750 may also include displaying other areas in a value between red and yellow by determining a threshold of each section according to examinations. The colors of red and yellow are selected at random for convenience of explanation, and other colors may also be used to output a graphic image of a heat distribution type.

In another exemplary embodiment, the diagnosis supporting apparatus 100 may output graphic image data of a contour type that is converted from an examination path by using one or more of the analyzed examination characteristics. When the examination path is acquired by using an ultrasound imaging device in correspondence with respective characteristics of an organ to be examined, the examination path may include data that relates to depth. The diagnosis supporting apparatus 100 may output graphic image data of a contour type by including data that relates to depth in characteristics of an examination conducted by a practitioner. For example, the diagnosis supporting apparatus 100 may determine a threshold according to depths, and may output each section by using a different color index that varies based on depths, in which among examination paths, the diagnosis supporting apparatus 100 may output a relatively deep examination path in red, and a relatively shallow examination path in yellow. The colors of red and yellow are selected at random for convenience of explanation, and other colors may also be used to output a graphic image of a heat distribution type.

In still another exemplary embodiment, the diagnosis supporting apparatus 100 may output an analysis point. For example, the diagnosis supporting apparatus 100 may compare a reference path included in reference examination information with an examination path included in characteristics of an examination conducted by a practitioner, in order to determine a section in which a difference therebetween is greater than a threshold upon comparison to be an analysis point, and the diagnosis supporting apparatus 100 may output the determined analysis point on a screen, and output results of a diagnosis that corresponds to the analysis point.

Further, based on the difference between the examination path and the reference examination information, the diagnosis supporting apparatus 100 may visually output characteristics of an examination. By comparing a reference path included in reference examination information with an examination path included in characteristics of an examination conducted by a practitioner, the diagnosis supporting apparatus 100 may determine a section based on a difference obtained from the comparison, and the diagnosis supporting apparatus 100 may visually output the examination path in a heat distribution type or a contour type. For example, even when the difference is not significantly above a threshold, the diagnosis supporting apparatus 100 may visually output the difference between a reference examination and an examination path. Further, the diagnosis supporting apparatus 100 may visually output a moving distance of an examination device, a moving speed in each section, an average moving speed, a period of staying in each area, an examination frequency in each area, and the like, based on the difference of each category. In this case, the diagnosis supporting apparatus 100 may output only the difference, or may output the difference by overlaying the difference on an examination path.

Further, a diagnosis supporting method performed by the diagnosis supporting apparatus 100 may further include automatically selecting a reference examination from among a plurality of reference examinations based on a practitioner's characteristics that include a competence of the practitioner, or based on a practitioner's input.

The selection of the reference examination at the diagnosis supporting apparatus 100 may include selecting a reference examination before a practitioner conducts an examination, and selecting/re-selecting a reference examination after a practitioner conducts an examination. The reference examination may be selected based on the purpose and need of examinations.

The methods and/or operations described above may be recorded, stored, or fixed in one or more transitory or non-transitory computer-readable storage media that includes program instructions to be implemented by a computer to cause a processor to execute or perform the program instructions. The media may also include, alone or in combination with the program instructions, data files, data structures, and the like. Examples of computer-readable storage media include magnetic media, such as hard disks, floppy disks, and magnetic tape; optical media such as CD ROM disks and DVDs; magneto-optical media, such as optical disks; and hardware devices that are specially configured to store and perform program instructions, such as read-only memory (ROM), random access memory (RAM), flash memory, and the like. Examples of program instructions include machine code, such as produced by a compiler, and files containing higher level code that may be executed by the computer using an interpreter. The described hardware devices may be configured to act as one or more software modules in order to perform the operations and methods described above, or vice versa. In addition, a computer-readable storage medium may be distributed among computer systems connected through a network and computer-readable codes or program instructions may be stored and executed in a decentralized manner.

A number of examples have been described above. Nevertheless, it should be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. A diagnosis supporting apparatus, comprising:
an examination path acquiring component configured to acquire an examination path of an examination device;
an examination characteristic analyzer configured to analyze at least one characteristic of an examination based on the acquired examination path by using reference examination information; and
a diagnosis component configured to determine an analysis point based on the reference examination information and the analyzed at least one examination characteristic, and to obtain a diagnosis based on at least one image that relates to the determined analysis point.

2. The apparatus of claim 1, wherein the at least one examination characteristic comprises at least one from among an examination path, a moving distance of the examination device, a moving speed in at least one section, an average moving speed, a period of staying in at least one area, an examination frequency in at least one area, a path difference between the acquired examination path and the reference examination information, and a difference between an examination frequency of the examination path with respect to at least a first section and an examination frequency that relates to the reference examination information with respect to the at least first section.

3. The apparatus of claim 2, wherein the examination characteristic analyzer is further configured to convert the examination path into a graphic image of a heat distribution type by using at least one of the at least one examination characteristic.

4. The apparatus of claim 1, wherein the diagnosis component is further configured to compare the acquired examination path with a reference path that relates to the reference examination information, and to determine the analysis point as a section in which a difference between the acquired examination path and the reference path exceeds a predetermined threshold.

5. The apparatus of claim 4, wherein the diagnosis component is further configured to calculate a distance between at least one point on the examination path and a predetermined point within the analysis point, and based on the calculated distance, to determine a point from among the at least one point on the examination path in order to obtain a diagnosis based on an image that relates to the determined point.

6. The apparatus of claim 1, wherein the diagnosis component is further configured to determine, as the analysis point, at least one from among a section in which a moving speed exceeds a predetermined first threshold, a section in which an average speed is greater than a predetermined second threshold, a section in which a period of staying in at least one area is shorter than a predetermined third threshold, and a section in which an examination frequency is lower than a predetermined fourth threshold.

7. The apparatus of claim 6, wherein the diagnosis component is further configured to extract a point at which respective examination paths intersect in the analysis point, and to obtain a diagnosis based on an image that relates to the extracted point.

8. The apparatus of claim 1, further comprising an output device configured to output at least one from among the at least one examination characteristic, the analysis point, and a result of the obtained diagnosis.

9. The apparatus of claim 8, wherein the output device is further configured to visually output, on a screen, the at least one examination characteristic based on a difference between the acquired examination path and the reference examination information.

10. The apparatus of claim 1, further comprising a reference examination selector configured to automatically select a first reference examination from among a plurality of reference examinations based on at least one from among an input received from a practitioner and a plurality of characteristics of the practitioner that include a competence of the practitioner.

11. A diagnosis supporting method, comprising:
acquiring an examination path of an examination device;
analyzing at least one characteristic of an examination based on the acquired examination path by using reference examination information;
determining an analysis point based on the reference examination information and the analyzed at least one examination characteristic; and
obtaining a diagnosis based on at least one image that relates to the determined analysis point.

12. The method of claim 11, wherein the at least one examination characteristic comprises at least one from among an examination path, a moving distance of the examination device, a moving speed in at least one section, an average moving speed, a period of staying in at least one area, an examination frequency in at least one area, a path difference between the acquired examination path and the reference examination information, and a difference between an examination frequency of the examination path with respect to at least a first section and an examination frequency that relates to the reference examination information with respect to the at least first section.

13. The method of claim 11, wherein the analyzing the at least one examination characteristic comprises converting the examination path into a graphic image of a heat distribution type by using at least one from among the at least one examination characteristic.

14. The method of claim 11, wherein the determining the analysis point comprises comparing the acquired examination path with a reference path that relates to the reference examination information, and determining the analysis point as a section in which a difference between the acquired examination path and the reference path exceeds a predetermined threshold.

15. The method of claim 14, wherein the obtaining the diagnosis comprises:
calculating a distance between at least one point on the examination path and a predetermined point within the analysis point;
based on the calculated distance, determining a point from among the at least one point on the examination path; and
obtaining the diagnosis based on an image that relates to the determined point.

16. The method of claim 11, wherein the determining the analysis point comprises determining, as the analysis point, at least one from among a section in which a moving speed exceeds a predetermined first threshold, a section in which an average speed is greater than a predetermined second threshold, a section in which a period of staying in at least one area is shorter than a predetermined third threshold, and a section in which an examination frequency is lower than a predetermined fourth threshold.

17. The method of claim 16, wherein the obtaining the diagnosis comprises extracting a point at which respective examination paths intersect in the analysis point, and obtaining a diagnosis based on an image that relates to the extracted point.

18. The method of claim 11, further comprising outputting at least one from among the at least one examination characteristic, the analysis point, and a result of the obtaining the diagnosis.

19. The method of claim 18, wherein the outputting comprises visually outputting, on a screen, the at least one examination characteristic based on a difference between the acquired examination path and the reference examination information.

20. The method of claim 11, further comprising automatically selecting a first reference examination from among a plurality of reference examinations based on at least one from among an input received from a practitioner and a plurality of characteristics of the practitioner that include a competence of the practitioner.
